(19) United States
(12) Patent Application Publication
(10) Pub. No.: US 2024/0115404 A1
(43) Pub. Date: Apr. 11, 2024

(54) ANTI-FOULING STENT

(71) Applicant: BVW Holding AG, Cham (CH)

(72) Inventors: Lukas Bluecher, Eurasberg (DE); Michael Milbocker, Holliston, MA (US)

(21) Appl. No.: 18/220,238

(22) Filed: Jul. 10, 2023

Related U.S. Application Data
(60) Provisional application No. 63/388,076, filed on Jul. 11, 2022.

(51) Int. Cl. A61F 2/82

(52) U.S. Cl. CPC A61F 2/82; A61F 2250/0026

(57) ABSTRACT

An endoprosthesis device comprising a tubular structure having an outer surface and an inner surface such that the inner surface locates a lumen, and wherein the outer surface includes a microstructure pattern having hierarchical microstructures generating an adhesive effect to a target surface. The inner surface comprises a microstructure pattern that is superhydrophobic or oleophobic that is capable of being anti-fouling wherein the microstructure pattern of the inner surface comprises microridges. Additionally, the outer surface may include pores that fluidly connect the outer surface to the inner surface for transporting fluid from the target surface interface into the endoprosthesis lumen.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application claims benefit of the following patent application(s) which is/are hereby incorporated by reference: 63/388,076 filed on Jul. 11, 2022

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

The present invention relates to gastrointestinal stents.

Stents are frequently used to enlarge, dilate or maintain the patency of narrowed body lumens. A stent may be positioned across a narrowed region while the stent is in a compressed state. The stent may then be expanded in order to widen the lumen. Stents used in the gastrointestinal system are commonly constructed of plastic or coated metal wire. Plastic and coated metal wire stents facilitate retrieval and/or replacement of the stent during a follow-up procedure.

However, plastic stents are not expandable like metal wire stents. That is, plastic stents have a fixed diameter. Since plastic stents are frequently delivered through the working channel of an endoscope, the diameter of the working channel limits the diameter of the stent. For example, plastic stents typically have a diameter that is no greater than 11.5 Fr (French catheter scale). However, such a small diameter stent rapidly becomes clogged within the biliary and pancreatic ducts, thereby requiring replacement every three months, or even sooner.

In the case of the coated metal wire stent, there is less limitation on the diameter of the stent when it is deployed since such stents can be compressed and then expanded at the delivery site. However, these stents, as well as the plastic stents, can have issues of migration from its target lumen site and leakage of fluids around the outer surface of the stent. In some instances, fluid accumulation between the outer surface of the stent and the target lumen surface can be the cause of stent migration, although there are also other causes for migration to occur as well. A device capable of preventing the buildup of fluids between the stent and the target lumen surface may resist migration of the stent due to fluid buildup.

Prior art stents have been designed to secure the drainage stent at a site of implantation within the body lumen including some mechanical mechanism for retaining the drainage stent within a body lumen. For example, retention flaps radially projecting from the tubular body have been used previously. Retention flaps may be formed by making an oblique slit along the length of the tubular member. Each slit defines a tab and enables the tab to project outwardly of the outer surface of the tube to engage the lumenal surface of the biliary duct to prevent migration. The tabs at the opposite ends of the drainage stent typically extend toward the middle of the stent as well as radially outward. The openings defined by the tab-forming skives may provide access to the interior of the stent where cellular or other material may tend to develop into an obstruction causing a restriction of flow through the stent.

Drainage stents in the prior art have also included one or more curled or coiled end portions. For example, the distal and/or proximal ends of a drainage stent may have a curled configuration, often referred to as a “pigtail” configuration. One such example is shown by U.S. Pat. No. 5,052,998 to Zimmon which discloses an indwelling drainage stent having flaps at one end, a series of drainage perforations along the length of the drainage stent and a pigtail configuration at the opposite end. Other stents include anchoring flaps or pigtail loops at both ends of the stent. Prior art stents have been provided both with and without the drainage perforations as shown in the Zimmon patent.

Other structures, such as helical tubular drainage stents having an inflatable portion, are disclosed by Rucker in US 2006/0167538A1, filed Dec. 21, 2005. Kolb describes drainage stents having one or more curled portions and a drainage channel having a laterally open portion in US 2006/0052879 A1, filed Aug. 16, 2005. It should be apparent from the previously mentioned prior art that stent geometry has been dramatically affected by the need to anchor the stent within the body lumen. Therefore, there is a need for decoupling the anchoring mechanism from the stent geometry of gastrointestinal stents so that the stent can be designed in a more advantageous and effective geometry.

Furthermore, insertion of a drainage stent placed by endoscopic sphincterotomy may require stretching and cutting of the Sphincter of Oddi and surrounding areas, which may compromise the function of the Sphincter of Oddi after insertion of the drainage stent. In addition to the sphincterotomy procedure for inserting a drainage stent, placement of the position of the biliary stent at the Papilla of Vater (e.g., across the Sphincter of Oddi) may also lead to duodenogastric reflux. A compromised Sphincter of Oddi may allow fluid flow in the reverse direction from the duodenum, or duodenogastric reflux, causing bacteria and biofilm deposition, and possibly occluding the biliary duct or the drainage stent. Thus, there is a need for a gastrointestinal stent which does not require stretching or cutting of the Sphincter of Oddi and surrounding areas.

Regarding biliary stents in particular, drainage devices may be implanted to treat various conditions. For example, drainage stents configured as biliary stents may be implanted in the biliary tract to treat obstructive jaundice. FIG. 1 is illustrative of a typical biliary system identifying a right hepatic duct joining with the left hepatic duct to form a common hepatic duct, further identifying a gallbladder and a cystic duct, a pancreas and a main pancreatic duct, and all aforementioned ducts connecting to form a common bile duct leading to a duodenum through the Ampulla of Vater and Sphincter of Oddi.

FIG. 2 is illustrative of a typical stent placement in the biliary system 200. The stent 201 may be placed in the gastrointestinal tract near the common hepatic duct 206 and the common bile duct 216 where it enters the duodenum 218.

As a surgical treatment for treating stricture or occlusion of a biliary tract, a biliary stent placement is known, in which a radially expandable biliary stent is implanted in a lesioned part of the tract. By performing the biliary stent placement, patency of the lesioned part in the biliary tract can be ensured and as a result, for example, remediation of obstructive jaundice symptoms can be achieved.

Biliary stents used in a biliary stent placement procedure conventionally have a tubular stent body configured to be expandable and a membrane extending to protrude from one end of the stent body in a cylindrical manner Conventional biliary stents are configured such that the membrane extends out toward the duodenum when implanted in a lesioned part of a biliary tract. The membrane is configured to allow bile outflow from a gallbladder to be directed toward the duodenum and also to prevent a backflow from the duodenum to the gallbladder. In this configuration, a function of allowing bile flowed out from the gallbladder to flow toward the duodenum and also preventing a backflow from the duodenum to the gallbladder is referred to as “valve function.”

The issues that occur in ductal obstruction and occlusion are unique to the biliary system due to the harsh environment of the biliary drainage system due to pancreatic and biliary enzymes, as well as microbes such as fungi and bacteria that inhabit the ducts make stasis and injury to these ducts susceptible to complete occlusion by biofilms and organic masses. Thus, there is a need for a biliary stent having an inner lumenal coating that discourages occlusion by these biofilm and organic masses so as to minimize or eliminate complications by occlusion of the stent lumen.

Further, bleeding of the ducts, whether due to stent placement or some other causation, can lead to clot formation and occlusion in and around the stent. Invasion by a tumor within the ductal wall and occlusion of the duct by external compression by a tumor mass can be common causes of complications. Therefore, there is a need for a biliary stent that may have a rigid expanded diameter without requiring a high normal force to be applied to the body lumen.

Treatment of ductal occlusion has been partially addressed with stainless steel stents, cobalt chromium stents, inexpensive plastic stents and, more recently, shape memory alloy stents. However, because these stents are placed in a harsh enzymatic environment, occlusion and re-occlusion occurs frequently.

It is desirable to place a tubular implant, such as a stent, at the point at which the biliary duct enters the duodenum. This particular opening widens as the biliary duct approaches the duodenum. As a result, the desired shape of an implant would be frustoconical, but such an implant inherently tends to migrate due to the uneven shape of the stent. Thus, there is a need for a biliary stent with a frustoconical profile which also prevents stent migration.

On the other hand, removable stents are desired, for example, in applications for treating benign disorders. Some applications of stents include use as a bridge to treatment and less of a palliative measure, due in part to improvements in some cancer therapies and other methods of treating malignant growths. However, efforts to improve removability have been at odds with at least some measures taken to reduce risk of stent migration. Improved stents that reduce trauma during stent removal and improve stent adhesion to the body lumen are needed.

Therefore, what is needed is an improved stent with, for example, improved resistance to migration, improved stent adhesion to the lumen wall, and/or improved removability while further preventing common issues that arise with prior art devices. Surface textures on stents have not been used in the gastrointestinal tract due to the issue of the highly lubricated environment which causes stent migration. However, the surface textures of the present disclosure employ a unique surface microstructure which utilizes the lubrication aspect of gastrointestinal lumen to achieve stent anchoring. In some aspects, the surface texture may be configured to create Wenzel-Cassie zones of interface between the stent and the body lumen.

### BRIEF DESCRIPTION OF THE SEVERAL VIEWS OF THE DRAWINGS

FIG. 1 is illustrative of a typical biliary system.

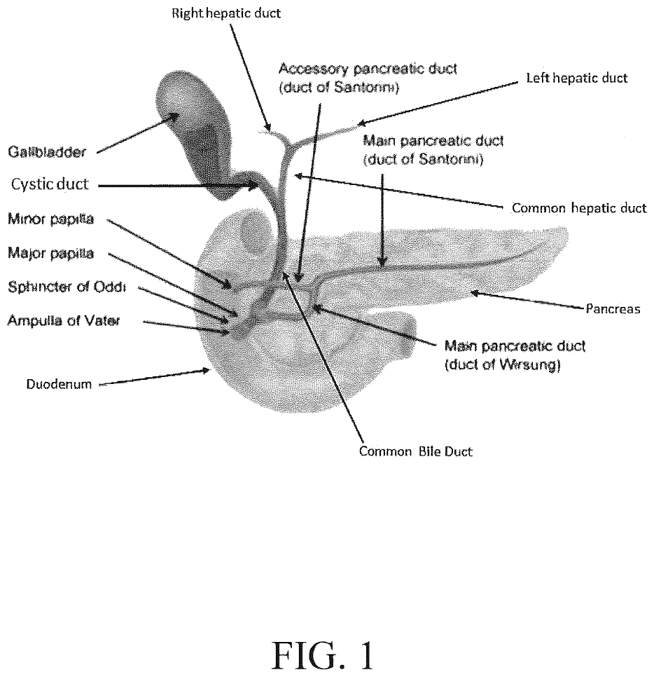

FIG. 2 is illustrative of a typical stent placement in the biliary system.

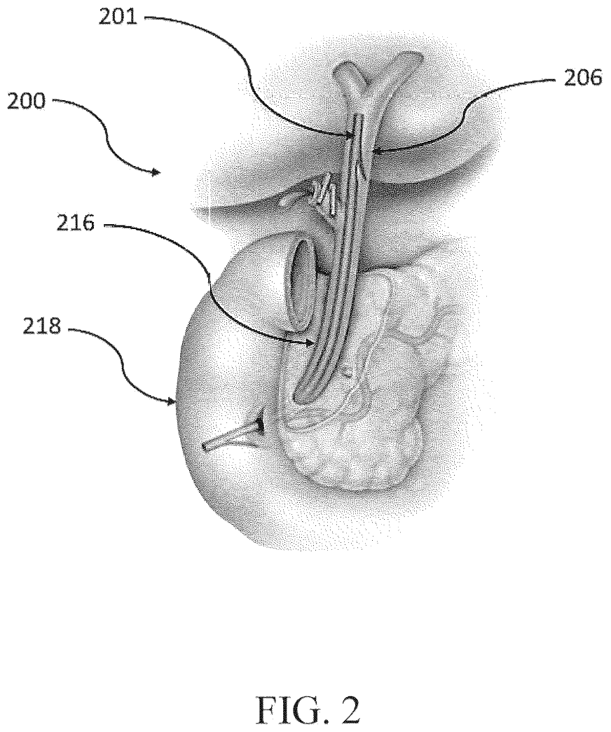

FIG. 3 is a cross-sectional view of an embodiment of a gastrointestinal endoprosthesis of the present disclosure. FIG. 3A is a side perspective view of an embodiment of the endoprosthesis.

FIG. 4 shows an antifouling microstructure.

FIG. 5A shows an antifouling microstructure isometric view, and FIG. 5B shows a side view of the antifouling microstructure.

FIG. 6 shows a gastrointestinal endoprosthesis, stent, of the present disclosure with fluid conductance means and a sloughing mechanism.

FIG. 7 shows an antifouling microstructure of the present disclosure.

### BRIEF SUMMARY OF THE INVENTION

The present disclosure provides an endoprosthesis that may comprise a polymeric coating. In some embodiments, the endoprosthesis may include a number of surface features such as protrusions or textures that are arranged in a micropattern. As used herein, a micropattern may include a regular or irregular array of micro-scale features (e.g., protrusions such as micropillars, voids such as textures). These micropatterns can be disposed hierarchically on the device.

A hierarchical structure may comprise a microstructure with a multi-scale morphology. Recent advances in nanomaterial science have made increasingly possible the design of hierarchical surfaces with specific and tunable properties. Most of this work has concentrated on hierarchical single-walled carbon nanotube films realized by a simple, rapid, reproducible, and inexpensive filtration process from an aqueous dispersion. By varying the thickness of carbon nanotube random networks using this filtration process, it is possible to tailor the wettability of microstructures based on capillary phenomena. This effect can be applied to porous films.

It has also been discovered that a multi-fractal structure, especially when constructed in a hierarchical surface morphology, may generate a unique two-dimensional extension of the Wenzel and Cassie-Baxter theories of surface wetting. Capillary phenomena in a film hierarchical-morphology may generate surprising adhesive characteristics. For example, the applicants calculate that the difference in the surface energy of a microstructured surface and a wet target, considered separately and when in contact, may increase proportionally to the number and size of juxtaposed hydrophilic and hydrophobic regions on microstructured surface and target surface. The larger the difference between the surface energies of the liquid free surfaces and solid contacting surfaces can provide a quantitative measure of adhesion. This difference in surface energies is referred to as the barrier energy, or the energy required to break an interface with this structure and allow translation.

The long-range ordering of water may be protected against thermal disruption by the establishment of Wenzel-Cassie zones of hydrophilic regions bounded by hydrophilic-hydrophobic boundaries. Furthermore, the hydrophobic regions may serve as ready reservoirs for displaced hydrophobic solutes, further reinforcing the structure and increasing the barrier energy. This effect may be particularly enhanced when the target surface is a biological tissue since the fluid at the interface between microstructure surface and a biological tissue is generally a suspension of lipids (hydrophobic) in water (hydrophilic).

In some embodiments, there may be a hierarchy of ordering, from the microscopic zones of water and lipid, to the exclusion of multi-molecular sized lipid particles, to the ordering of individual water molecules, and so on. Hence, a microstructure surface that is also hierarchical, and ordered on at least two-dimensional scales is reasonably expected to enhance the localizing effects of generating a high barrier energy.

The present disclosure may include devices that utilize liquid interfaces between an endoprosthesis, e.g., a stent, and a target surface, i.e., lumen of a tissue to 1) immobilize the endoprosthesis on a target surface, 2) create a fluidic seal between endoprosthesis and target surface, and/or 3) present an anti-fouling surface that either a) resists deposition of a solid state on the endoprosthestic surface or b) promotes a deposition that is inherently unstable and wicks off in a predictable and intended way.

Immobilization, sealing, and anti-fouling functionality of devices disclosed herein may be caused to originate in the surface energy modification of the disclosed surfaces. Hydrophilic-hydrophobic interfaces, such as those developed in the Wenzel-Cassie interface state, may play a role more profoundly than generally assumed in the prior art. Solutes in aqueous suspension are extensively excluded from the vicinity of many interfaces, and such exclusion arises from long-range restriction of water molecules, nucleating at the interface of a macrostructure surface and projecting well into the aqueous phase, similar to what occurs in liquid crystals. The presence of such unexpectedly large zones of mobility-limited water may impact many features of surface and interfacial chemistry.

The present disclosure further relates to a device including adhesive surfaces bearing a microstructured surface wherein the microstructured surface comprises at least two kinds of features. Further, said adhesive surfaces may comprise features wherein the lateral aspect ratio of the features range from about 0.1 to about 50 for each feature, and at least one feature dimension varies by at least a factor of 10%. For example, in one embodiment, two sets of pillars may be present, one set being 5 microns in diameter and 30 microns tall and another set of pillars at least 15 microns in diameter and 75 microns tall, wherein the first set of pillars is disposed on the top surface of the second set of pillars. At least two of the feature dimensions (height, width, and/or length) may be microscopic. In some embodiments, all three of the feature dimensions (height, width, length) may be microscopic.

In at least one embodiment, an endoprosthesis has optionally an expanded state and a contracted state, and in some cases may include a stent with a polymeric coating attached (e.g., adhered, etc.) to an outer surface of the stent. The stent may have an inner surface defining a lumen. The stent may have an outer surface and a stent thickness defined between the inner surface and outer surface. The stent may comprise a plurality of surface textures extending from the stent surfaces, wherein the textures are arranged in a micropattern. The micropattern may reside on the exterior surface and/or the interior surface, or both. The micropattern on the exterior surface may anchor and/or seal the stent to the target surface. The micropattern on the interior surface may resist fouling. In at least one embodiment, the stent may be a flared stent. Optionally, the stent may include surface features that conduct fluid transport wherein fluid is conducted from the space between the exterior prosthetic surface and the target lumen surface, or fluid transport means where fluid may be transported to the lumen of the stent. The latter may be achieved with through-holes which may generate a flow gradient due to capillary action. In some embodiments, the through-hole may have a uniform passage, such as a cylinder. In some embodiments, the through-hole may be tapered, or may be frustoconical in shape.

In one or more embodiments, the stent may include a base and a tissue engagement portion. The base may include a first surface (e.g., attached or comprising an outer surface of the stent). The tissue engagement portion may include a second surface facing outwardly from the stent. The tissue engagement portion may include a structure that defines a plurality of protrusions or wells extending outwardly/inwardly from the second surface away/toward the base. In at least one embodiment, the surface textures may be arranged in a micropattern. In one or more embodiments, the base and the stent may be coterminous. In one or more embodiments, the base may cover any apertures of the stent. When the endoprosthesis is expanded or in the implantation configuration in a lumen defined by a vessel wall, the structure defining the plurality of surface textures may generate an adhesive force that is capable of creating an interlock between the vessel wall and the endoprosthesis. The interlock may be immobilizing and/or fluidically sealing.

In one or more embodiments, the stent surface may include a plurality of protrusions (e.g., micropillars) of at least two scale dimensions extending from the base (e.g., outwardly from the stent) and arranged hierarchically. In one or more embodiments, the protrusions may be arranged in a regular micropattern (e.g., of micropillars). Exterior surface microstructure may be capable of 1) generating axially directed shear force to inhibit axial migration of the stent, or 2) generating a radially directed normal force to attract the target lumen surface to the exterior stent surface to generate a sealing means, or both.

In one or more embodiments, the immobilization and sealing capability may together or individually introduce a locking capability of the device to a target surface, wherein the exterior microstructure of the device changes dimension due to absorption of fluid when placed in the target lumen, and this fluid absorption causes the dimensions of the microstructure to change and create a grasping action. For example, microstructure pillars may expand to trap tissue disposed in the interstices between pillars.

In one or more embodiments, the stent surface may include a plurality of fluid conductive microstructures designed to transport liquid in an intended direction. Typically, fluid may be transported axially along the exterior surface of the stent to remove fluid from the space between the exterior stent surface and the target surface. Alternatively, fluid may be transported radially through holes in the stent wall from the interface volume to the lumen of the stent. The latter fluid transport feature may be used in biliary stents, since such stents often cross over bifurcation of a tissue lumen, wherein it is desirable that the cross lumen is permitted to drain into the lumen of the stent.

In one or more embodiments, the stent surface may include a plurality of protrusions of at least one scale dimension extending from the base inwardly toward the lumen of the stent that effectively presents an anti-fouling surface that may be capable of preventing accumulation of fluid constituents conducted through the stent lumen.

In one or more embodiments, the stent surface may include a plurality of protrusions of at least one scale dimension extending from the base inwardly toward the lumen of the stent that may provide a surface that promotes deposition of a certain morphological type. For example, the microstructures may be arranged as circular islands capable of promoting discontinuous deposition, which is easily sloughed away as particulate.

Alternatively, an absorbable surface coating on the microstructure may provide for sloughing of a deposited layer.

Although not wishing to be bound by theory, a target tissue may engage a micropatterned surface or coating via one or more non-mechanical mechanisms. As disclosed herein, in certain embodiments of this disclosure, non-abrasive engagement may be achieved via creation of one or more interface structures such as, but not limited to, 1) Wenzel-Cassie, 2) capillary, and/or 3) eigenmode or Schallamach.

In some embodiments, the target tissue may interlock with a micropatterned coating having one or more microstructures by tissue encroachment around and/or between the one or more micropillars. In at least one embodiment, a tissue grasping mechanism may result in tissue engaging and/or interlocking with a micropatterned coating having a structure defining one or more textures (e.g., voids, negative spaces, etc.) or networks of connected textures, wherein tissue and/or cell ingrowth occurs within the textures.

In one or more embodiments, a van der Waals bond mechanism may be formed between a tissue in contact with a micropatterned coating that may include, for example, a mucoadhesive gel.

In one or more embodiments, engagement of tissue with a micropattern having an appropriate geometry may be by proximity attraction by van der Waals bonding. Herein, “interlock” may be understood to refer to engagement of a target tissue by a microstructure having micropillars and/or micro-holes via any one or more of the mechanisms (e.g., tissue ingrowth, chemical bond, proximity attraction, etc.) described herein or otherwise known to one of skill in the art.

However, some embodiments of the present disclosure may not rely on tissue engagement via grasping or friction. Indeed, initial tissue engagement mechanisms may enable localization of the tissue to the device by the surface energy properties of the microstructures of the present specification. Surface energy interactions, which may be mediated by capillary bridges, may be characteristically nondestructively reversible, such that the stent can be removed from the body lumen without damage to the lumenal tissue.

The applicants have surprisingly found that surface structures comprising hydrophilic and hydrophobic domains in juxtaposition, either laterally or in a stacked fashion, may attract or repel different intrabody fluid and solid constituents. For example, the hydrophilic regions of the surface texture may associate with hydrophilic tissue constituents, and hydrophobic regions of the surface texture may associate with hydrophobic constituents. During this association, the interface energy may decrease, resulting in stent-tissue adhesion. Disruption of this adhesive state may require the expenditure of energy, and hence Wenzel-Cassie associations may be stable to dislocation. Even when micro-dislocation occurs, the Wenzel-Cassie state may be quickly re-established.

It can be appreciated that in certain embodiments, van der Waals forces may participate in the formation of Wenzel-Cassie zones of adhesion, but they do not result in denaturation of proteins, which may be understood to characterize the usual bonding mechanism disclosed herein. In the Wenzel-Cassie zone of interaction, hydrophilic and hydrophobic zones may be interlocking, and displacement of the stent may require mixing these zones, which may require energy input.

In a second unexpected aspect, the applicants have discovered that capillary action can play a role in stabilizing the Wenzel-Cassie zones, as well as providing an initial mechanical aspect which may draw together stent and lumen surfaces. For example, wells or through-holes disposed on the stent surface may employ the difference in surface tension between hydrophilic and hydrophobic domains, as well as the surface energy of the base material, to transport liquid during the formation of the stent-lumen interface. For example, if the lumen surface is eluding a liquid, the surface texture of the stent may be configured to induce capillary domains that draw this fluid away from the stent-lumen interface.

It should be appreciated that a hydrophilic/hydrophobic condition can be induced on a surface either 1) chemically or based on the molecular structure and/or 2) texturally, induced by the geometry and hierarchical arrangement of microstructure. The hydrophobicity of both surface types may typically be characterized by surface energy.

Lastly, the applicants have discovered that target surfaces may exhibit characteristic spatial frequency responses. This property of materials may be referred to as the eigenmode response of the surface. In the present context of this disclosure, eigenmode response may be the inducement of a surface wrinkle in the target surface with a characteristic spatial frequency. Matching the spatial frequency of the textured surface of the stent to the eigenmodes of the lumen surface can substantially stabilize an adhesive interface. In particular, matching the spatial frequency of the textured surface across several spatial eigenmode frequencies of the target surface by a hierarchical arrangement of the surface texture may result in a multiplicity of interface zones that form zones of energy minima Disrupting these energy minima costs energy, and hence the stent may be stabilized against migration without unwanted forces acting on the tissue surface (such as those that cause inflammation, cell death, or cellular injury).

It should be appreciated that a Wenzel-Cassie interface may be considered a multidimensional low energy state that juxtaposes attractive and repulsive forces. Surface domains may be attractive and/or repulsive due to a variety of factors. Those factors may include, but are not limited to, ionic content of the interface layer, the degree of order or disorder in the material comprising the surface pattern, and various chemical treatments of surfaces. Consequently, as used herein, Wenzel-Cassie interfaces may include any interface where attractive and repulsive domains are formed between a textured surface and a target surface in a low energy state. The difference between the interface energy state and the energy states of the textured and target surfaces in separation may be linearly proportional to the strength of adhesion in shear. To achieve high peel force (normal force adhesion) one may employ capillary aspects into the surface pattern design.

As disclosed herein, combinations of the above-described discoveries may result in an adhesive textured surfaces that can be optimized to a variety of target surfaces. One, experienced in the art of solid-state physics, can measure the energy difference between the separate and joined states of textured surface and target surface to consider how to design certain surfaces disclosed here.

In addition to any adhesive characteristics of the devices disclosed herein, there may also be a need for a repulsive surface to prevent stent fouling after deployment into the tissue lumen. Such a surface may employ a globally hydrophobic surface, or a zoned hydrophobic surface, in which alternating zones of hydrophobicity and hydrophilicity are capable of preventing biofilm formation and/or the migration of fungus or bacteria. In one embodiment, the interior stent surface may be a modulated hydrophobic/hydrophobic surface, and the exterior surface may be a modulated hydrophobic/hydrophilic surface, wherein each surface may be described as a hierarchical patterned surface designed to a specific biologic interface.

The micropattern may be specifically designed for a particular tissue to effectively localize the stent to the target tissue. In at least one embodiment, the micropattern may be present along at least a portion of the endoprosthesis. In at least one embodiment, the texture of the micropattern can be uniform or the micropattern can be formed of texture having a first configuration and texture having a second configuration.

The shape of at least some of the plurality of textures may be selected from a group including cylinders, rectangular prisms, polygonal prisms, spheres, spheroids, ellipsoids, and similar shapes. Some textures may be continuous and selected from a group including sinusoidal height variation (waves), ridges, concentric structures, and similar shapes.

In at least one embodiment, the textures of the micropattern may be at least two types of cylindrical pillars, each cylindrical pillar having a diameter and a height, wherein the diameter of each cylindrical pillar may be equal to 0.1 to 1 times its height. The pillars may be stacked hierarchically and arranged to an offset rectangular grid pattern and disposed on a sinusoidally varying substrate.

In at least one embodiment, each texture of the micropattern has a first dimension and a second dimension, wherein the first dimension may be between about 1 micron and 1000 microns (e.g., between about 1 micron and 100 microns), wherein the second dimension may be between about 1 micron and 1000 microns (e.g., between about 10 microns and 150 microns), and wherein one texture may be entirely disposed on top of the other texture, wherein a ratio between the pitch of the first texture and the pitch of the second texture may be between about 0.1 and 0.5. In at least one embodiment, each protrusion has a ratio between the first dimension and the second dimension that may be between about 0.2 and 0.3.

In at least one embodiment, the endoprosthesis may be retrievable by, for example, a retrieval loop at a distal end of the stent.

In at least one embodiment the endoprosthesis may be distally anchored by one or more pigtail configurations of the stent.

Several methods of manufacturing an embodiment of the endoprosthesis are known in the art. One method of manufacturing includes forming a polymeric coating, wherein the polymeric coating includes a base and a tissue engagement portion. The base includes a first surface. The tissue engagement portion includes a second surface facing away from the first surface and includes a structure that defines a plurality of textures extending inwardly or outwardly from the second surface toward the base. In one or more embodiments, the textures are arranged in a micropattern. The method further includes providing a stent having an inner surface defining a lumen and an outer surface; and attaching the base of the polymeric coating to either the outer surface of the stent, the inner surface of the stent, or both.

In one or more embodiments, the micropattern of textures may be made using lithography techniques, salt leaching, electrospinning, and/or laser ablation. In some embodiments that include a micro pattern of micro pillars, the polymeric coating can be formed using a mold having an inverse of the micropattern and injecting a polymeric material into the mold and, in some cases applying temperature or pressure to the mold, before the polymeric material cures, using soft lithography techniques, or by etching the polymeric coating from a layer of the polymeric material. In at least one embodiment, an adhesive layer is applied to at least one of the surfaces of the base and the outer surface of the stent. In at least one embodiment, the polymeric coating is formed as a tubular structure. In one or more embodiments, the polymeric coating is formed in a strip, which is wrapped (e.g., helically wrapped, circumferentially wrapped, randomly wrapped, etc.) about the outer surface of the stent.

In at least one or more embodiments, the polymeric coating is disposed on the stent so as to allow a portion of the stent to move relative to the target tissue. This feature is particularly important when the target lumen generates a peristaltic motion. A combination of “slip” and “stick” features of the proper spatial frequency can allow the stent to transmit peristaltic motion without being displaced by such motion. For example, circumferential rings of textured surface may be placed on the stent at a spatial frequency that is a prime fraction of the peristaltic spatial frequency. For example, if the spatial frequency of the peristaltic motion is one, then the rings could be placed at ⅓ that spatial frequency. Alternatively, one end of the stent may be without surface texture and the other end with surface texture, such that compressional pressure between the two ends is defeated by slippage of one end.

The present disclosure may include embodiments that include a textured surface stent device, which may allow for sufficient anchoring while reducing the risk of migration, reducing fluid accumulation between the exterior stent surface and the target lumen, and additionally provide an interior microstructured surface that inhibits fouling of the surface.

The present disclosure may include embodiments including a stent that is adapted both to resist migration within a body lumen and to conform to a tortuous installation path or installed location. The stent may include a first section, typically self-expandable, of predetermined compressibility adapted to permit the section to conform to the shape of the body lumen through which it is transmitted or surrounding the section when deployed. The stent also may include a second section which may be free to move within the target lumen.

An endoluminal stent graft may include one or more segments of a healing promoter attached within a proximal anchor region of an endoluminal stent graft, and, optionally, within one or more distal anchor regions. The healing promoter may be a chemical disposed in an absorbable layer, or simply a second textured surface designed to direct a specific type of cellular ingrowth. The healing promoter may be a tissue scaffold. When the endoluminal stent graft is positioned within a lumen, the segments of the healing promoter promote and guide the migration, proliferation and adhesion of vessel cells to the endoluminal stent graft to increase localized healing. Thus, healing time after implant of an endoluminal stent graft may be decreased and a more stable implant produced that is less susceptible to migration and/or endoleaks that could otherwise form at the sides of the proximal neck.

### DETAILED DESCRIPTION OF THE INVENTION

The following detailed description and appended drawings describe and illustrate various exemplary embodiments of the invention. The description and drawings serve to enable one skilled in the art to make and use the invention. Although the present invention will be described with reference to preferred embodiments, those skilled in the art will recognize that changes may be made in form and detail without departing from the spirit and scope of the invention. As such, it is intended that the following detailed description be regarded as illustrative rather than limiting and that it is the appended claims, including all equivalents thereof, which are intended to define the scope of the invention.

The term “proximal” as used herein is a broad term and is to be given its ordinary and customary meaning to a person of skill in the art and refers without limitation to a direction that is generally towards a physician during a medical procedure.

The term “distal” as used herein is a broad term and is to be given its ordinary and customary meaning to a person of skill in the art and refers without limitation to a direction that is generally towards a target site within a patient's anatomy during a medical procedure.

The terms “comprise(s),” “include(s),” “having,” “has,” “contain(s),” and variants thereof, as used herein are broad terms and are to be given their ordinary and customary meaning to a person of skill in the art and are intended to be open-ended transitional phrases, terms, or words that do not preclude the possibility of additional acts or structure.

The term “body lumen” as used herein is a broad term and is to be given its ordinary and customary meaning to a person of skill in the art and refers without limitation to a body passage cavity that conducts fluid, including but not limited to gastrointestinal tract, biliary ducts, pancreatic ducts, ureteral passages, esophagus, and blood vessels such as those of the human vasculature system.

The term “implantable” as used herein is a broad term and is to be given its ordinary and customary meaning to a person of skill in the art and refers without limitation to an ability of a medical device to be positioned at a location within a body, such as within a body lumen. Furthermore, the terms “implantation” and “implanted” as used herein are broad terms and are to be given their ordinary and customary meaning to a person of skill in the art and refer without limitation the positioning of a medical device at a location within a body, such as within a body lumen.

The terms “endolumenal,” “intraluminal,” and “transluminal” as used herein are broad terms and are to be given their ordinary and customary meaning to a person of skill in the art and refer without limitation to implantation procedures wherein the medical device is advanced within and through a body lumen from a remote location to a target site within the body lumen. Endolumenal delivery can include implantation in a biliary duct from an endoscope or catheter.

The term “expandable mesh” as used herein is a broad term and is to be given its ordinary and customary meaning to a person of skill in the art and refers without limitation to self-expanding and/or non-self-expanding configurations made of any generally rigid or elastic material which when expanded have an open network or arrangement which would otherwise allow tissue in-growth and would not otherwise prevent fluid flow through its walls. Several prior art mesh stents have been utilized with a polymeric sheath or cover; however, these sheaths must be stretched to increase in size. These materials exert a force that resists expansion, which tends to limit the final expanded size of a prior art mesh. Additionally, this resistance may make expansion of the mesh more problematic. Alternatively, the sheath may be folded or bundled over the prior art mesh when it is compressed, so that no force is exerted upon expansion. However, this method increases the overall size of the compressed prior art stent, so that a larger size catheter is required for a given size stent.

The term “hierarchical” as used herein is a broad term and is to be given its ordinary and customary meaning to a person of skill in the art and refers without limitation to microstructures having dimensions that are, in at least one aspect, larger and smaller in relation to each other, i.e., a larger dimension microstructure and a smaller dimension microstructure. In some examples, the larger dimension microstructure may support the smaller dimension microstructure. Generally, a micro-scale feature will be understood to include a structure having a dimension (e.g., length, width, height, pitch, and/or slope) in a range of from about 1 micrometer to about 10,000 micrometers. Herein, unless the context indicates otherwise, micro-scale features may collectively be referred to as a surface texture.

The term “tissue adhesive” as used herein is a broad term and is to be given its ordinary and customary meaning to a person of skill in the art and refers without limitation to a surface comprising a hierarchical micropattern which, when in contact with a target surface can resist translation in a direction orthogonal to the target surface and/or parallel to the target surface. As an example, a device that is tissue adhesive can generate a peel force and/or a shear force, when placed in contact with a target surface and acted upon by some external force.

The term “cell promoter” as used herein is a broad term and is to be given its ordinary and customary meaning to a person of skill in the art and refers without limitation to a surface that can direct cells in a particular direction, and/or promote a certain type of cell to populate a surface, and/or direct a certain combinations of cells to populate a surface, and/or promote certain cell types while blocking other cell types. In one example, a cell promoter can cause tissue that is adjacent to the cell promoter surface to heal faster and with more strength than some other tissue that is not adjacent to the cell promoter.

The term “anti-fouling surface” is a broad term and is to be given its ordinary and customary meaning to a person of skill in the art and refers without limitation to a surface that can resist the accumulation of a molecule, a particulate, or a cell. As an example, an anti-fouling surface can be a surface which inhibits the accumulation of matter from the environment in which the surface is placed.

The term “Wenzel interface” is a broad term and is to be given its ordinary and customary meaning to a person of skill in the art and refers without limitation to a surface having a surface texture made of a plurality of surface features that when placed in contact with a wet target surface can draw water in between the surface features of the surface texture.

The term “Cassie interface” is a broad term and is to be given its ordinary and customary meaning to a person of skill in the art and refers without limitation to a surface having a surface texture made of a plurality of surface features that when placed in contact with a wet target surface prevents water from interpenetrating between the surface features of the surface texture.

The terms “eigenmode”, “wrinkle eigenmode” and “wrinkle mode” are broad terms and are to be given their ordinary and customary meaning to a person of skill in the art and refer without limitation to a natural wrinkling of a tissue surface when exposed to a shear force. Tissue eigenmodes are well defined and characterized by the flexural modulus of the tissue. A given tissue type can naturally wrinkle, or develop a spatial periodicity, characterized by a spectrum of these spatial periodicities.

The terms “Schallamach wave” and “Schallamach wrinkle” are broad terms and are to be given their ordinary and customary meaning to a person of skill in the art and refer without limitation to an occurrence of waves of detact ent known to occur during abrasion experiments with target substrates. In some examples, Schallamach waves are explained in terms of the elastic instability of the electromeric surface. For example, if a surface of a device is designed to anticipate these waves by employing a surface periodic structure which can be slightly lower in spatial frequency than the anticipated Schallamach wave, then the Schallamach wave can catch in the periodic structure and dramatically increase the shear force required for translation. Such a “Schallamach-matching” design can educe abrasive damage between the device surface and target surface.

The terms “micropattern” or “microstructure” are broad terms and are to be given their ordinary and customary meaning to a person of skill in the art and refer without limitation to textures (e.g., stent textures) of a regular or irregular pattern wherein the shortest center-to-center distance (i.e., the distance between the geometric centers, pitch) of adjacent textures (i.e., textures that share a side) is greater than 1 micrometer. In a regular pattern of textures, each of the two geometric centers are equidistant from the side shared by the adjacent textures. In an irregular pattern of textures, the two geometric centers are not equidistant from the side shared by the adjacent textures. In an irregular pattern of textures, the spacing parameters may satisfy a mean value, for example an average distance between microstructure centers.

The phrases connected to, coupled to, and in communication refer to any form of interaction between two or more entities, including mechanical, electrical, chemical, magnetic, electromagnetic, fluid, and thermal interaction. Two components may be coupled to each other even though they are not in direct contact with each other. For example, two components may be coupled to each other through an intermediate component.

Various medical devices for implantation in a body lumen are disclosed herein. Some embodiments relate to a medical drainage device comprising two or more planar curvilinear bends in a tubular member, where each bend can be curved in opposite directions with respect to adjacent bends. For instance, a pair of consecutive planar curvilinear bends may form a pigtail shaped or spiral configuration. In some embodiments, the curvilinear bends can define a tortuous portion of the drainage lumen within the tubular member.

The medical drainage devices disclosed herein may be described with respect to an exemplary biliary stent embodiment comprising a tubular support member. However, the embodiments of biliary drainage stents can also illustrate other drainage devices, such as ureteral stents, esophageal stents or drainage catheters provided in accordance with other embodiments. For example, a drainage stent could be configured for use within a ureteral, urethral, esophageal or blood vessel.

While the subject matter of the present disclosure may be embodied in many different forms, herein there are described in detail various embodiments of the present disclosure. This description is an exemplification of the principles of the present disclosure and is not intended to limit the present disclosure to the particular embodiments illustrated.

For the purposes of this disclosure, like reference numerals in the figures shall refer to like features unless otherwise indicated.

In some embodiments, the present disclosure relates to micropatterned polymeric coatings or surfaces for use on medical devices. In some embodiments, the micropatterned polymeric surfaces may be utilized with implantable medical devices, such as stents, to reduce or prevent stent migration, particularly for stents used in the gastroesophageal system, including, but not limited to, esophageal, biliary, and colonic stents. In one or more embodiments, the micropatterned polymeric coating may include regularly or irregularly spaced microscale textures. In one or more embodiments, the micropatterned polymeric coating may include regularly or irregularly shaped microscale textures (e.g., voids, spaces, channels, passages, etc.). In one or more embodiments, the micropatterned polymeric coating may include regularly or irregularly spaced and shaped microscale textures. In some embodiments, such microscale textures may promote, for example, controlled cell migration and tissue ingrowth.

The microstructures of the present invention may be capable of generating forces due to: 1) phobic/philic effects, 2) microtopology, 3) absorption chemistry, 4) morphological swelling, and/or 5) active mechanical interaction. These forces may be generated in dry conditions, damp conditions, wet conditions, or a combination thereof.

In some embodiments, the devices of the present disclosure may include an anti-fouling aspect, wherein microstructures exhibiting oleophobic properties on the inner surface(s) of a stent can contribute to antifouling properties. In certain embodiments, the antifouling surface may comprise pores which can attract a film of water creating a super-nano-hydrophilic structure to form a protective layer which may exhibit anti-fouling properties.

In addition to antifouling properties, some devices may be capable of generating a similar effect of self-cleaning. Self-cleaning strategies do not necessarily inhibit deposition, and rather may present surface topographies similar to sharkskin or butterfly wing microstructure, that are known to cause accumulated materials to be easily released from the microstructured surface.

In some embodiments, the micropatterned surfaces can be modified with zwitterionic polymer brushes or with polyelectrolyte multilayers to enhance their antifouling and/or self-cleaning properties. Additionally, in some embodiments anti-fouling properties can be combined with a fluid transport functionality. For example, surfaces with microscopic ridge-like morphology can exhibit fluid transport and anti-fouling properties.

Anti-fouling can be associated, in some embodiments, with drag reducing microstructured surfaces. For instance, sharkskin morphology is known to have anti-fouling characteristics. One interesting aspect of sharkskin microstructure related to anti-fouling capability is its ability to promote lamellar flow. Turbulent flow across a surface is understood to cause fouling as a result of re-entrant fluidic circuits that can roll along the interface surface. Some microstructure surfaces can generate a hydrodynamic lifting effect similar to the aerodynamic flow of air over an airplane wing. While fluids cannot expand like a gas in the example of an airplane wing, the lifting effect of the microstructure can push nucleation particulates away from the surface, creating a fluid zone devoid of particulates.

In one preferred embodiment, the stent comprises regions of 1) immediate adhesion to a target lumen, 2) sealing to a target lumen, 3) fluid transport and/or 4) anti-fouling.

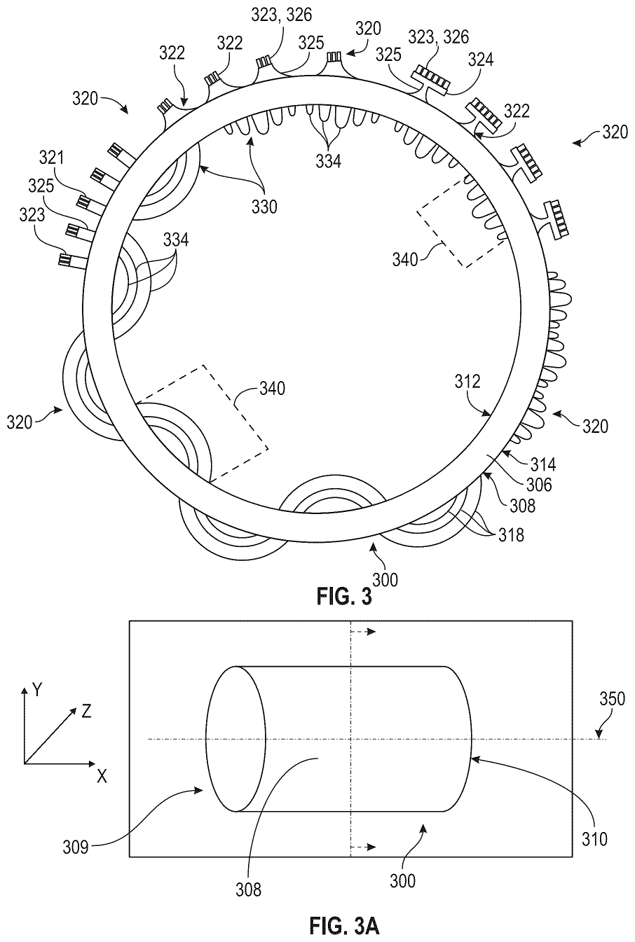

An embodiment of an endoprosthesis as disclosed herein is illustrated in FIG. 3, having a lateral cross-sectional view of the endoprosthesis. As shown in FIG. 3, the endoprosthesis 300 can include a polymeric wall 306. In one or more embodiments, an endoprosthesis 300 can be a preformed stent. In certain embodiments of this disclosure, an endoprosthesis 300 may include a tubular member 308 which may have at least a portion including a constant diameter, may include one or more tapers, or one or more flares, and/or other changes in diameter in the tubular member 308 and/or at one or more ends 309, 310. The endoprosthesis 300 depicted in FIG. 3 may include an inner surface 312 and an outer surface 314 of the polymeric wall 306. The outer surface 314 may include one or more microstructure patterns disposed about the surface. In some embodiments, the hierarchical microstructure pattern 320 of the present invention on the outer surface 314 may cover only a portion of the outer surface. In some embodiments, the hierarchical microstructure pattern 320 may cover from 1% to 100% of the outer surface 314. In some embodiments, the hierarchical microstructure pattern 320 may cover 5%, 10%, 15%, 20%, 25%, 30%, 35%, 40%, 45%, 50%, 55%, 60%, 65%, 70%, 75%, 80%, 85%, 90%, 95%, or 99% of the outer surface 314. In other embodiments, as illustrated in FIG. 3, the outer surface 314 may include multiple microstructure patterns disposed about the surface such that the surface is covered from 1% to 100% by the multiple patterns. It will be easily understood that the percentage of each microstructure pattern disposed about the outer surface 314 may be within the recited percentage's identified above, together totaling to 100%, or less.

In some embodiments, a microstructure pattern 320 of the outer surface 314 may comprise hierarchical microfeatures 321 wherein a smaller microfeature 323 is disposed about a larger microfeature 325. In some embodiments, the microstructure pattern 320 may comprise hierarchical microfeatures 323, 325 wherein the larger microfeature 325 includes recurved pillars 322. In some embodiments, the recurved pillars 322 may include a T-shaped configuration 324 at its top section. In certain embodiments, the recurved pillars 322 may include smaller pillars 326 disposed thereon. In some embodiments, the smaller pillars 326 may be disposed about the top surface of the recurved pillars 322. In some embodiments, recurved pillars 322 may be arranged in a triangular pattern with respect to adjacent pillars. The recurved pillars 322 may be arranged such that the pitch, or spacing, between adjacent pillars may be from 1-500 microns, from 1-250 microns, from 25-250 microns, or from 25-100 microns. In some embodiments, the recurved pillars 322 may have a diameter from 1-500 microns, from 1-250 microns, from 1-100 microns, from 1-50 microns, or from 10-50 microns. In some embodiments, the recurved pillars 32 may have a height from 1-500 microns, from 1-250 microns, from 1-100 microns, from 1-50 microns, or from 10-100 microns. In certain embodiments, the recurved pillars 322 may have pitch, or spacing, between adjacent pillars from 25-100 microns, a diameter from 10-50 microns, and a height from 10-100 microns. For embodiments wherein the recurved pillars 322 comprise a T-shaped top configuration, the top portion of the T-shape configuration can be circular with a diameter from 1.2-2.5 times the diameter of the base portion of the recurved pillar.

In some embodiments, the second pillars 326 disposed about the recurved pillars 322 may have a pitch between adjacent pillars from 10-50 microns, a diameter from 10-150 microns, and a height from 1-10 microns. These second pillars 326 may be made of a liquid swellable polymer in some embodiments.

In certain embodiments, a target tissue may be retained between the large microfeatures 322, 325. In certain embodiments, the tissue may be retained between the T-shaped configuration 324 of the recurved pillars 322. In some embodiments, the T-shaped configuration 324 may have swelling characteristics which can provide a pincer action on or around the tissue 328, whether in contact or non-contact.

In at least one embodiment of this disclosure, the outer surface 314 may include a microstructure pattern for transporting fluids.

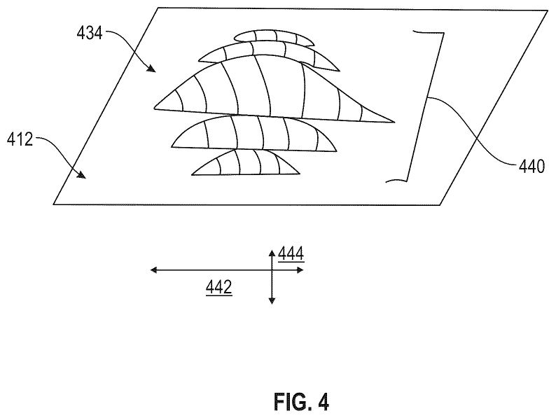

As shown in FIG. 3, the inner surface 312 may include a microstructured pattern 330 comprising microridges 334 which may be made of microfeatures having a ridge like shape. In certain embodiments, the microridges 334 may include an air-foil like profile wherein the ends have a tapered shape, and the central portion is taller than the end portions. The microstructured pattern 330 may include multiple microfeatures of the microridges 334 which may be spaced with their edges separated between adjacent features from 1 nm to 100 microns, from 10 nm to 50 microns, or from 100 nm to 10 microns (See FIG. 4 for additional illustrations, microridges 434). As shown in FIG. 4, in some embodiments, the microridges 434 may have a length in the axial dimension 442 that may be longer than the width 444 of the microridge 434. In certain embodiments, the ridges 334, 434 may form a grouping 340, 440, or island, wherein the single microfeatures may be considered a first microstructure level, and the grouping or island shapes 340, 440 may be considered a second microstructure level. The two microstructure levels can be expected to work together to promote lamellar flow through the stent lumen.

In some embodiments, the microstructured pattern 320 of the outer surface 314 may include microfeatures in the form of microridges 318 for fluid conduction properties. The microridges 318 may have a pitch from 1-500 microns, from 1-250 microns, from 1-100 microns, from 1-50 microns, or from 5-50 microns. The microridges 318 may include a width from 1-100 microns, 1-50 microns, 1-40 microns, 1-30 microns, or 1-20 microns. The microridges 318 may include a height from 1-500 microns, from 1-250 microns, from 1-100 microns, from 1-50 microns, or from 5-50 microns. With reference to FIG. 3A, the microridges 318 may be arranged such that the length of the microridges 318 are positioned in a circumferential pattern in relation to the central axis 350 of the endoprosthesis (i.e., in the “y” axis shown in FIG. 3A), or the microridges 318 may be arranged such that the length of the microridges are positioned axially in relation to the central axis of the endoprosthesis (i.e., in the “x” axis shown in FIG. 3A). The plurality of microridges 318 may be parallel, convergent, or bifurcating. In certain embodiments the bifurcation of the microridges may be directed towards the end of the stent to wick fluid away from the interface volume delimited by the outer wall of the stent and the surface of the target lumen.

In at least one embodiment, shown in FIG. 4, the inner surface 412 may include at least one pattern of microridges 434. In some embodiments, the inner surface 412 may include a second pattern of microridges that may include additional plurality of microridges. As disclosed above, but illustrated in a different manner, in some embodiments, the length dimension 442 of the microridges 434, may be oriented in an axial position in relation to the central axis of the endoprosthesis. In some embodiments, the length 442 of the microridges 434 may be oriented in a circumferential pattern in relation to the central axis of the endoprosthesis. In yet other embodiments having a plurality of microridges 434, a first portion of a plurality of microridges may be oriented in an axial configuration and a second portion of a plurality of microridges may be oriented in a circumferential configuration in relation to the central axis of the endoprosthesis. The microridges 434 may have an airfoil profile that is capable of directing particulate away from the stent substrate surface 412.

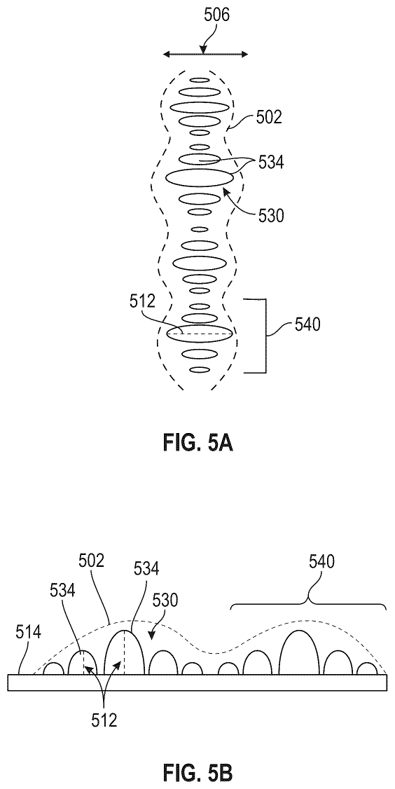

Referring now to FIG. 5A, in certain embodiments, the microstructure pattern 530 of the inner surface may be arranged with a sinusoidal distribution 502 with respect to the varying lengths and/or heights of the microfeatures (shown in FIG. 5B). The groupings of microstructures 540, or the islands as referenced above, may be comprised of microridges 534 where the grouping or islands 540 may be considered a first microstructure level and the microridges 534 may be considered the second microstructure level. The sinusoid arrangement 502 is illustrated in an “aerial view” (shown in FIG. 5A) and in a “side view” (shown in FIG. 5B) as an illustrative reference. The center position 512 of an individual microridge 534 may be spaced equidistant in a variety of geometrical patterns, such as a square grid pattern or triangular pattern. Additionally, the microridges 534 can be arranged in a uniform array or a non-uniform array. Further, the groupings or islands 540 may be arranged in a similar manner wherein the groupings are arranged in relation to one another in a geometric pattern, such as a square or grid pattern, or in a uniform or non-uniform pattern. Additionally, referring to both individual microridges 534 and groupings/islands 540, one subset may be arranged in a geometric manner, or uniform or non-uniform manner, while another subset may be arranged differently, and so on for other subsets.

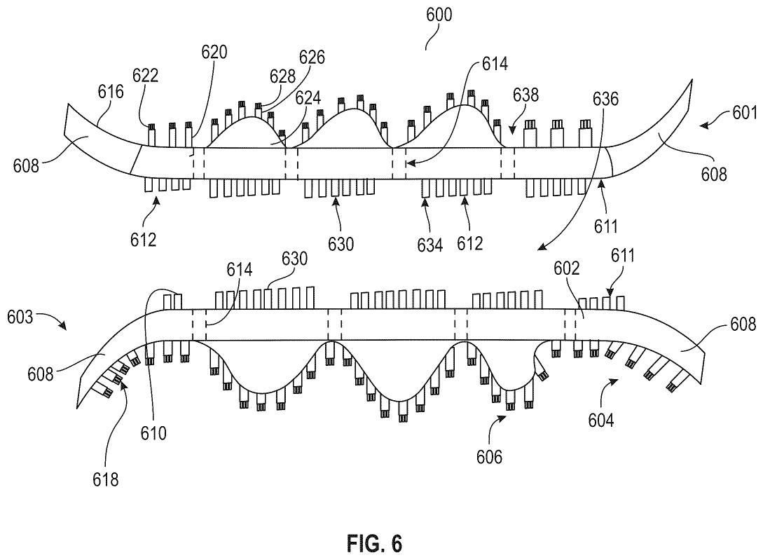

Referring now to FIG. 6, an embodiment is illustrated comprising a longitudinal cross section view of an endoprosthesis device 600 having a substrate 602 comprising an outer surface and inner surface, wherein the outer surface comprises external sealing microstructures 604. As shown in FIG. 6, the endoprosthesis 600 can have a first end 601 and a second end 603. In some embodiments, the external sealing microstructures 604 may be hierarchical having at least a first microstructure disposed about a second microstructure in a hierarchical manner, as previously disclosed elsewhere herein. In some embodiments, the endoprosthesis device 600 may further include anti-migration microstructure pattern 606. In some embodiments, the device 600 may include flared ends 608. In some embodiments, the device 600 may include an anti-fouling microstructure pattern 610 and 612 disposed about the inner surface 611. In certain embodiments, the endoprosthesis device 600 may include a fluid conduction microstructure pattern 614. In other embodiments, the endoprosthesis device 600 may include a combination of different microstructure patterns, including a sealing microstructure pattern 604, anti-migration microstructure pattern 606, anti-fouling microstructure pattern 610, 612, and a fluid conduction microstructure pattern 614. In yet another embodiment, the device 600 may include any combination of the foregoing microstructure configurations.

As depicted in FIG. 6, the external surface 616 of the flared ends 608 may be smooth or may be ornamented with a microstructure pattern 618. In some embodiments, external microstructure pattern 604 may comprise a sealing configuration, such as sealing microstructure 604 identified previously, and may be comprised of first pillars 620 having a diameter from 10-100 microns, a pitch from 20-200 microns, and a height from 10-100 microns. In some embodiments, the sealing microstructure may further include second pillars 622 disposed about the first pillars 620 wherein the second pillars may include a diameter from 1-10 microns, a pitch from 2-20 microns, and a height from 1-10 microns.

In some embodiments of the device 600 comprising an anti-migration microstructure pattern 606, the microstructure pattern may comprise a first two-dimensional sinusoidal microstructure 624 which may have a peak to peak distance (pitch) from 50-1000 microns and peak height from 50-2000 microns. An anti-migration microstructure pattern 606 as disclosed herein may further comprise a second set of microstructures 626 which may have a diameter from 10-100 microns, a pitch from 20-200 microns, and a height from 10-100 microns. Further, an embodiment of the anti-migration microstructure pattern 606 disclosed herein may include a third set of microstructures 628 which may have a diameter from 1-10 microns, a pitch from 2-20 microns, and a height from 1-10 microns. In certain embodiments, an anti-fouling microstructure pattern 610 may include nanoparticulate 630 randomly dispersed about the surface of the device. In certain embodiments, the nanoparticulate 630 may be dispersed about the inner stent surface 61. In some embodiments, circularly arranged pillars disposed about the inner surface 634 may have a diameter 1-10 microns, pitch 2-20 microns, and height 1-10 microns that act as nucleation islands that cause deposition that preferentially flakes off in response to fluid flow in the stent lumen 636. Fluid conduction microstructure 614 may comprise through-holes which conduct fluid from outer stent surface 638 to stent lumen 636.

In certain embodiments, the anti-migration microstructure pattern 606 may also include first microstructure 624 peak to peak distance chosen to match an eigenwrinkle spacing suitable for mating a shear force induced target surface wrinkle in the space between peaks of microstructure 624.

The anti-fouling nanoparticulate 630 may range in size from 1 to 100 nanometers and may present a hydrophobic surface. The size of the microfeatures 628 can be chosen to be more oleophilic with respect to the nanoparticulate surface 630, such that deposition may be caused to accumulate in circularly arranged islands of the microfeatures 628.

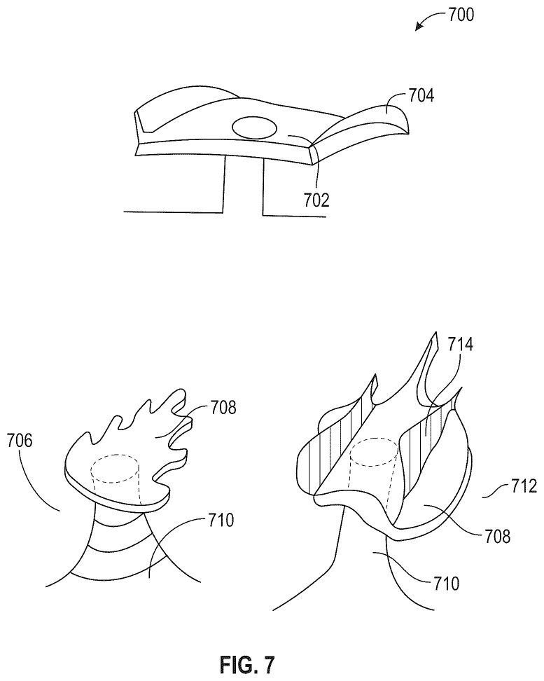

Referring now to FIGS. 7A, 7B, and 7C, three embodiments are illustrated with respect to an antifouling microfeature 700. In one or more of these embodiments, the microstructure surface may create a hydrodynamic effect capable of pushing away particulates entrained in a fluid flow. In FIG. 7, top illustration, the microstructure 702 may comprise a top portion having an airfoil design with upturned wing tips 704 positioned on one or more distal ends of the top surface. The wing tips 704 may be configured to reduce turbulent flow of fluid along the microstructure. In FIG. 7, bottom left, the microstructure 706 may include a configuration that mimics sharkskin which may comprise a top portion 708 having a serrated edge attached to a flared base pillar 710. In some embodiments, the top portion 708 may be planar or curvilinear. In some embodiments, the top portion 708 may comprise a central portion having a bulk that is thicker than the bulk of the edges. In FIG. 7, bottom right, the microstructure 712 is a combination of the foregoing embodiments wherein the top portion 708 may include a serrated edge along with a set of microstructures of fins 714 projecting outwards from the top portion surface.

In one or more embodiments, one or more textures may extend completely through the thickness of the coating. In one or more embodiments, one or more of the textures is a blind texture (e.g., a cavity, an indentation, a texture having a bottom, a texture that does not extend from the second surface to the first surface).

In some embodiments, as shown in FIGS. 3-6, the micropillars may be of any geometry, including, but not limited to, cylinders, prisms with a rectangular or polygonal base, pyramids, bumps, squares, elliptical, and the like. In some embodiments, the micropillars may be a combination of the foregoing arranged hierarchically. Hierarchical arrangement results in an overall texture which may be compound and non-traditional in shape with a plurality of protuberances, valleys, and/or ridges on multiple surfaces that do not define a cross-section that is circular, square, polygonal, etc. Individually, the textures can be micropillars with, for example, a circular cross-section, square cross-section, rectangular cross-section, star-shaped cross-section, hexagonal cross-section, pentagonal cross-section, heptagonal, octagonal cross-section, nonagonal cross-section, decagonal cross-section, other polygonal cross-sections, or non-traditional shaped cross-sections. In some embodiments, the cross section of any particular type of texture structure may have less of an effect on the characteristics of the surface as compared to surface energy domains created by the juxtaposition and stacking of different texture structures.

It will be understood by those skilled in the art that the structural dimensions (height, width, diameter, pitch, slope, and length) may be considered when designing surfaces to produce a desired effect. One or more of the microstructure textures as described elsewhere herein may have a cross section with a first dimension “h” that may be the greatest distance between the outer surface of the base and the end of the structure, and a second dimension “d” that is the greatest distance between two opposite sides (e.g., of a pillar). In some embodiments, “h” may relate to the “height” of the structure, and “d” may relate to the “diameter” of the structure. In some embodiments, “h” may be the overall height, or “h” may refer to a particular length of a component of the structure. The same may be true for “d”, where the diameter may be the overall diameter of the structure, or may be a particular diameter of a component of the structure. For example, for an embodiment having a circular cross-section the second dimension may be the diameter. For a square cross-section, the diameter may be measured from two opposing sides. For a rectangle, the major dimension may be between two shorter opposing sides. For a star cross-section, the major dimension may be measured between two opposing points. And for a hexagon cross-section, the major dimension may be between two opposing points. In some embodiments, the second dimension “d” may be between midpoints of two opposite sides. In at least one embodiment, a cross section of a micropillar taken in the radial direction has at least four sides.

Embodiments of the present disclosure contemplate polygonal cross-sections which may include all sides of equal length, or may include combinations of sides of equal length and unequal length, or may include all sides of unequal length. Embodiments of the present disclosure contemplate multiple pillars of multiple cross-sectional shapes including traditional shapes (e.g. circles, squares, rectangles, hexagons, polygons, etc.) and non-traditional shapes having a perimeter where at least a portion of the perimeter is curvilinear. In at least one embodiment, the micropillars may be solid structures. In other embodiments the micropillars may be partially solid structures. In yet other embodiments, the micropillars may be hollow structures. In at least one embodiment, each micropillar may have a constant cross-section, but in other embodiments the micropillars may have variable cross-sections. In at least one embodiment, a micropillar structure may extend perpendicularly from the base. In at least one embodiment, a micropillar structure may extend from a base in a non-perpendicular angle wherein the geometric center of the end of the micropillar may be offset laterally from the geometric center of the area of the base covered by the micropillar. For example, a longitudinal axis of the micropillars extending through the geometric centers of the lateral cross-sections may form an angle that is less than 90 degrees with the base. In at least one embodiment, the plurality of micropillars can be arranged in a hierarchical arrangement in one or more particular micropatterns.

In one or more embodiments, textures may take any of the shapes and dimensions described elsewhere herein. In general, a hierarchical arrangement may include a base structure. In some embodiments, the base structure may be flat, may be continuously varying as in a sinusoidal profile, may be stepped, for example, as an ascending and descending staircase, may be perforated, or otherwise varied in a random or regular pattern. For example, in a sinusoidal profile in two dimensions, the pattern may be characterized by a wavelength or range of wavelengths. One aspect of the present invention is understood to include a second set of textures that may be disposed on top of the base structure. The second texture may be arranged as a pillar, a ridge, a pyramid, and/or the like, as listed previously. This second set of textures also may include a characteristic dimensional measure, which may include pitch, height, diameter, width, slope, and the like. In certain embodiments, the dimensional measure of the base may be larger than the dimensional measure of the second set of textures. In some embodiments, a ratio between first and second measure is between 10 and 0.5. In certain embodiments, there may be a third set of textures disposed on the second set of textures, and optionally disposed on the first set of textures located between the structures of the second set of textures. Depending on the desired effect, the ratio between these dimensional measures may be adjusted to produce the desired effect. However, it will be understood that simply “adjusting” the dimensional measures to produce a desired effect is not a trivial task, and one must undertake thorough experimentation to establish the dimensional measures to produce the desired effect. One cannot simply select a variety of features and simply produce a desired result. Small changes in one dimensional measure or in a different geometry can have undesirous effects on the characteristics of the microstructure surface.

For embodiments where microstructures may produce antifouling effects, the pitch of adjacent microstructures relative to their height, e.g., pillars, may be relatively small. For example, the height of the microstructures may be 1 to 10 times the pitch between the microstructures. A higher height to pitch ratio tends to make a surface more hydrophobic and with low surface energy. Low surface energy structures may tend to resist deposition of ionic moieties that may be typically found in biological tissue. If the desired effect of a microstructure surface is to promote cellular migration along the surface, then the structural dimensions may be chosen to generate a surface energy gradient along the surface. Such structural dimensions and positions may be designed such that cells can readily bridge and travel along the structures, and thereby migrate across the surface. In contrast, microstructure surfaces with deep valleys disposed about the surface may tend to discourage cell migration. It will be appreciated that cells depend on a continuity of attachment sites in order to propagate along a surface. Therefore, a combination of tall pillars spaced closely together (i.e., a small pitch), such that cells do not readily fit between the adjacent pillars, can have a desired effect of promoting cellular propagation across the surface without the cells growing into the surface. It will be appreciated that cellular ingrowth into a stent surface may be particularly discouraged due to complications and issues arising with such scenarios. Instead, a microstructure surface having the proper dimensions and positions of the structures can cause cells to be directed to a tissue defect for cellular repair without cellular ingrowth which can result in making stent removal difficult. In some embodiments, a stent may be textured so as to resist migration in relation to the deployment site. Migration resistant surface textures may typically include Wenzel-Cassie wetting states. Such surface textures may tend to create zones of hydrophilic attraction and hydrophilic repulsion in close proximity to each other, thereby creating forces that resist migration of the stent.

In certain embodiments disclosed herein, a device of the present invention may be a wire woven stent. In some embodiments, the spacing of the wires in relation to one another may match an eigenmode of the tissue that the stent will be deployed to. The eigenmode may be a flexural eigenmode or may be a peristaltic eigen mode, or both. When one or more of the eigen modes are excited by deployment of the stent, then an anti-migration coating may deform naturally to engage the target eigenmode(s). In some embodiments, the stent structure may be configured such that it induces a target microstructure in the polymer coating, then the coating can be made thinner, and enable the stent to deploy more easily when expanded.

In some embodiments, intimate contact between the surface texture and target lumen surface is desirable. In some embodiments, a gap between the surface texture and target surface may be desirable. In certain embodiments, the gap may, on average, be less than approximately 2 times the height of the largest surface texture height. If there is a fluidic or gaseous gap between the surface texture and target surface, the adhesion of the device to the surface is not dependent upon frictional engagement or any interlocking mechanism between the microfeatures (e.g., micropillars, textures, etc.) of the device and the target tissue. Instead, there is a fluid dynamic engagement between the surfaces that creates the anti-migration effect. Conversely, if frictional engagement with tissue is desired, such functionality can be added to the surface patterns of the present specification by designing at least a portion of the surface features to provide such engagement. For this reason, in at least one embodiment, one or more particular microstructures are selected that has a micropattern geometry and dimensions suitable for a particular application (e.g., anti-fouling, promotion of biological tissue formation, desired anti-migration properties, etc.).

In one embodiment, the micropillars in the micropattern may all have the same shape, and in other embodiments, the micropillars may vary in shape along the polymeric coating, along a portion of the polymeric coating, or in discrete portions of the polymeric coating. Thus, in at least one embodiment, the micropattern can include portions where the micropillars have a first configuration and portions where the micropillars have a second configuration. Moreover, embodiments may include the polymeric coating having only one micropattern or the polymeric coating having multiple micropatterns. Thus, the polymeric coating can be tailored to specific structural characteristics of the body lumen and a desired characteristic while using a single stent.

Similarly, in one or more embodiments, textures may be configured and arranged in the same manner described herein for micropillars disposed on a two-dimensionally sinusoidally varying base. That is, in at least one embodiment, the textures in the micropattern may have a discrete shape combined with a continuous shape, and in other embodiments, the textures may vary in shape along the polymeric coating. Thus, in at least one embodiment, the micropattern can include portions where the textures have a first orientation and portions where the textures have a second orientation. Moreover, embodiments include the polymeric coating having only one micropattern (e.g., of random textures, of micropillars, etc.) or the polymeric coating having multiple micropatterns (e.g., two or more different micropatterns of textures, two or more micropatterns of micropillars, one or more micropattern of textures in combination with one or more micropatterns of micropillars). Thus, the polymeric coating can be tailored to specific structural and/or anatomical characteristics of the body lumen (e.g., a vessel, etc.) and a desired frictional engagement or interlock can be achieved, while using a single stent. In one or more embodiments, a micropattern may include one or more textures in combination with one or more micropillars (e.g., a micropattern including a first number of textures alternating with a second number of micropillars, etc.). In one or more embodiments, a polymeric coating may include a micropattern of micropillars and a micropattern of textures, wherein the micropatterns may or may not overlap.

In some embodiments, the ends of the protrusions, such as micropillars, that are furthest away from the outer surface of the base can be shaped to improve tissue adhesion. In one or more embodiments, the ends can be tapered, pointed, rounded, concave, convex, jagged, and/or frayed. The ends of each second protrusion can also include a plurality of first pillars on an even smaller scale than the second micropillars.

Similarly, in some embodiments, the second surface of the tissue engagement portion can be adapted (e.g., shaped, textured, modified, etc.) to improve tissue adhesion. In one or more embodiments, the lateral and/or bottom surfaces of the textures can be tapered, fluted, punctured, concave, convex, jagged, and/or frayed.

In at least one embodiment, the protrusions can also include terminal features such as mushroom shaped terminations with involuting curvature, downward pointing spines, and/or a plurality of bumps with concave centers extending outwardly from a surface of the micropillar. In some embodiments, the terminal surface may include a plurality of indentations extending inwardly from a surface of the micropillar, a plurality of ridges arranged concentrically on a terminal surface of the micropillar, a tip at or near the end of the protrusion that is either softer or more rigid than the remainder of the protrusion, a frayed tip, a convex tip, a flared tip, a concave tip, a tip having a first dimension that is greater than a the diameter of the micropillar column extending outward from the base and the tip, and/or other features that may be coated with a thin layer of material with a specific surface energy, which may be useful in differentiating the surface energy of an adjacent surface such that a Wenzel-Cassie domain is established which is useful in gripping, improving stiffness, and/or flexibility characteristics for the interface between the device and tissue, and any combination of features thereof.

In at least one embodiment, the tip may be comprised of a different material than the remainder of the protrusion. Similarly, the end and/or lateral surfaces of textures may be shaped to improve tissue adhesion similar to that described above with respect to micropillars. For example, textures may include features such as smooth surfaces, rough surfaces, a plurality of bumps extending outwardly from a surface of the texture to create a capillary action aspect, a plurality of indentations extending inwardly from a surface of the texture, a plurality of ridges on a surface of the texture, a frayed end, a convex top end, a flared bottom end, a concave top end, a bottom having a first dimension that is greater than a characteristic diameter of a protrusion extending between the second surface and the end, and/or other features that may impact a flow aspect useful in developing a capillary suction aspect for gripping, making stiff the interface between device and tissue, or improve flexibility characteristics for the device by providing a gliding or flowing aspect on a microscale, and/or any combination of features thereof.

In at least one embodiment, the micropattern may include microridges and/or textures spaced apart (pitch) equidistantly of a first distance in the micropattern in a first region(s) of the base and spaced apart equidistantly of a second distance (which may be different than the first) in the micropattern in a second region(s) of the base. In at least one embodiment, the micropattern is a curvilinear array. In at least one embodiment, the micropattern of a first curvilinear array of microstructures may intersect at an angle with a second curvilinear array of microstructures to form a grid pattern (e.g., a square array). In at least one embodiment, the micropattern of microstructures is a regular n-polygonal array (e.g., hexagonal array), wherein a micropillar or texture may be present in the center of the microstructure forming polygons or may not be present in the center of a polygon in the center of the microstructure forming polygons. In other words, in the micropattern, the micropillars and/or textures are arranged in an array in the micropattern of the microstructure, wherein the rows and columns of the array may or may not be perpendicular.

In one or more embodiments, each micropillar or texture may have a longitudinal axis and the micropillars may be axially aligned in at least one of the axial directions (e.g., arranged in a row parallel to a longitudinal axis of the device) and the circumferential direction of the device (e.g., arranged in a row extending circumferentially around a longitudinal axis of the device). In at least one embodiment, the micropattern of micropillars or textures includes any or all of the features described in the previous paragraph.

In some embodiments the micropattern may cover only a portion of the base rather than the entire base. The micropattern of micropillars or textures may be helically disposed on the base. In one or more embodiments, a first micropattern may be disposed longitudinally along the base and a second micropattern is disposed circumferentially about the base so that the micropattern forms a tessellation-like configuration. Micropillars may be arranged in a row (e.g., parallel to a longitudinal axis of a stent), may be arranged in multiple rows which are continuous rows or discontinuous rows (e.g., aligned row segments separated by a gap). Discontinuous rows (and circumferentially oriented columns) can extend across the tessellations wherein the length of the discontinuity is five times the separation distance.

With regard to the material used for the polymeric coating, it may be useful that the material have a variety of characteristics that are useful for the environment it is created in and deployed in. For example, the material may be flexible and/or elastic enough to create a malleable contact with the tissue, be able to withstand the processing for creating the polymeric coating, and/or to accommodate stent mechanics such as elongation and conformability to tortuous anatomy. Examples of materials to be considered include, but are not limited to, flexible silicones, polyurethanes, hydrogels, mucoadhesive substrate, pressure-sensitive adhesives, and other suitable elastomers, such as synthetic rubbers. In some embodiments, stiffer substrates can be used in discrete configurations. Examples of acceptable stiff materials include, but are not limited to, polypropylene, polylactic acid polymer, PEEK, and polyacrylics.

In one or more embodiments, a coating having a micropattern may include and/or be formed from a biologically-derived protein structure (e.g., collagen, etc.). Other acceptable materials may include any flexible, biocompatible, and non-biodegradable polymer. For palliative treatment stent applications, it may be useful for the coating to include one or more non-biodegradable polymers and/or a material having a degradation profile that may be useful for the particular stent application and implantation site. In one or more embodiments, the coating may be biodegradable in order to, for example, allow stent removal (e.g., after some portion or all of the coating has degraded). Applications in which it may be useful to remove a stent include support during perforation healing, dilatation of benign structures, and bridge to surgery.

In at least one embodiment, the polymeric coating having micropillars and/or ridges may include polymerized hyaluronans capable of conforming to a lumen wall in a biochemical manner Hyaluronans are also capable of swelling in a liquid environment. Microstructure devices comprised of hyaluronan may be able to grasp target tissue in a pincer motion due to microstructure swelling.

In at least one embodiment, the polymeric coating may include at least one therapeutic agent. In other embodiments, an additional coating may be applied to the polymeric coating wherein the additional coating includes a therapeutic agent. Whether part of the polymeric coating, or an additional coating, a therapeutic agent may be a drug or other pharmaceutical product such as non-genetic agents, genetic agents, cellular material, protein removed extracellular matrix, etc. Some examples of suitable non-genetic therapeutic agents include but are not limited to, anti-thrombogenic agents such as heparin, heparin derivatives, vascular cell growth promoters, growth factor inhibitors, paclitaxel, etc. Where an agent includes a genetic therapeutic agent, such a genetic agent may include but is not limited to, DNA, RNA, mRNA, siRNA and their respective derivatives and/or components, especially where such genetic derivatives are bonded to a polymeric surface. Therapeutic agents may also include cellular material, wherein the cellular material may include but is not limited to, cells of human origin and/or non-human origin as well as their respective components and/or derivatives thereof. In one or more embodiments, a suitable therapeutic agent may include small organic molecules, peptides, oligopeptides, proteins (e.g., “hedgehog” proteins, etc.), nucleic acids, oligonucleotides, genetic therapeutic agents, non-genetic therapeutic agents, vectors for delivery of genetic therapeutic agents, cells, and/or therapeutic agents identified as candidates for vascular treatment regimens, etc., and combinations thereof.

In one or more embodiments, one or more therapeutic agents may be included within or on polymeric coating, including the micropillars and/or ridges. In some embodiments, plant derivatives such as terpenes, especially triterpenes, various acids, especially boswellic acid, and various phenols and antioxidative plant derivatives may by used as a therapeutic agent(s).

In one or more embodiments, the base may be formed from the same material as the micropillars and/or the structure of the tissue engagement portion. In one or more embodiments, the micropillars and/or structure are formed from one material and the base is formed from a different material. In one or more embodiments, the micropillars and/or structure are formed with layers of material, and these layers can be the same material or can be different materials depending on the characteristics required for the desired interaction of the device with the target tissue. Since differences in surface energy between adjacent hierarchical sites may play a role in establishing the Wenzel-Cassie interfaces of the present invention, differences in surface energy can be enhanced by coating some microstructure tips, or domain walls, with a thin layer of material having a desired ionic content. Chemical coatings may be used in place of a surface texture, since from a surface energy perspective, surface texture and surface chemistry may be understood as interchangeable.

Devices of the present disclosure may be understood to possess less abrasive localizing interaction with the lumen wall of the target tissue when inserted into a lumen, as compared to similar devices/stents relying on frictional engagement with the tissue. Consequently, removal of the devices disclosed herein may be easier with traditional removal techniques. In at least one embodiment, the device may be provided with a suture or removal loop on one end. In at least one embodiment, the removal loop may be provided on a distal end of the device. It should be noted that references herein to the term “distal” are to a direction away from an operator of the devices of the present disclosure, while references to the term “proximal” are to a direction toward the operator of the devices of the present disclosure. While sutures or removal loops are well known in the art for removing endoprostheses, sutures or removal loops have been provided on the proximal end of the stent, in other words the closest end to the practitioner. Here, the suture or removal loop is applied to the opposite end of the endoprosthesis. In at least one embodiment, the practitioner may grab the loop by placing the practitioner's tool inside and through the lumen of the endoprosthesis, grasping the loop on the distal end, and then by applying an axial force to the loop, the distal end of the endoprosthesis may be folded inward and pulled through the lumen of the endoprosthesis itself (i.e., device inversion). Thus, with such a method of removal, the micropillars may be peeled away from the vessel wall while the stent is flipped inside out to remove the endoprosthesis. This removal technique may be desirable since the surface textures of the present disclosure may have a reduced adhesion in peel force (perpendicular displacement) compared to surface adhesion in shear force (parallel displacement). In other embodiments, the practitioner may grab the loop from outside the endoprosthesis or at an end of the endoprosthesis.

To manufacture the endoprosthesis, several methods can be employed. The polymeric coating can be formed (e.g., molded) separately from the stent (e.g., as a polymeric film, a hydrogel film, a thin fibrous network, etc.) and then adhered to the stent (e.g., an outer surface of the stent) with an optional adhesive layer disposed between the outer surface of the stent and the base (e.g., the first surface) of the polymeric coating (e.g., applied to at least a portion of one or both of the first surface of the base and the outer surface of the stent). The polymeric material can be injected into a mold with the inverse of the micropattern to create the polymeric coating having a micropattern of microfeatures (e.g., micropillars, textures, etc.). Also, the polymeric material can be extruded through a mold using a vacuum pump system. In at least one embodiment, the polymeric coating can be created using soft lithography techniques.

In one or more embodiments, etching techniques can be used to create the coating, wherein material is taken away from a layer of the coating material to create the micropattern of the polymeric coating. In yet another embodiment, a technique called hot embossing can be used, which involves stamping partially cured polymer into the desired shape of the polymeric coating and then curing it before it is applied to the stent. Stamping may or may not include the use of a solvent. In one or more embodiments, a stent may be coated by any suitable method (e.g., spraying, dipping, injection molded, etc.), followed by the introduction of textures into the coating after the stent coating. In some embodiments, a fibrous network with micro-scale textures (e.g., voids) may be formed by electrospinning one or more fibers on a pre-coated stent. In one or more embodiments, a laser ablation process may be used, using one or more appropriately sized laser beams (e.g., same or different sizes depending on the desired pattern) to remove material from a coating in order to form one or more micropillars and/or one or more microridges.

In one or more embodiments, one or more portions of the coating may be deployed into a body lumen separately from a stent (e.g., as one or more pads, etc.). Then, for example, a gluco-adhesive may be applied to an applicable portion of a stent meant to attach to the pre-deployed coating (e.g., biointeractive pads). The radial expansive force of the stent during and after deployment may activate the adhesive and adhere the stent to the coating previously deployed in a body lumen. The gluco-adhesive aspect may be an alginate salt. The alginate salt on a smooth surface may dissolve into the tissue volume, and hence its adhesive effectiveness would be temporary. However, such an adhesive disposed on valley sections of a hierarchical texture may serve two purposes: 1) immediate adhesion, and 2) the mucoadhesive serves as a medium that can reinforce domain walls between hydrophilic and hydrophobic regions. Gluco-adhesives may be capable of reinforcing natural Wenzel-Cassie boundaries, giving a structural aspect to the domain walls rather than simply surfaces of equi-potential. Gluco-adhesives may in effect solidify initially established Wenzel-Cassie domains.

In one or more embodiments, a polymeric coating having negative textures (e.g., microholes) may be formed by using a technique called particulate dissolution (e.g., salt dissolving), wherein a composite material is formed from one or more polymeric materials and one or more particulates (e.g., soluble salts), followed by dissolving the one or more particulates (e.g., salts) from the composite material (e.g., with a solvent) resulting in a composite and/or polymeric material having textures or voids where the one or more particulates (e.g., salts) were removed. The salt can be an alginate salt, which provides capillary effect properties, using ionic concentrations to draw liquids into the voids formed by dissolution.

In one or more embodiments, a polymeric coating having a plurality of textures may be formed by a technique called electrospinning (e.g., using an electrical charge to draw very fine fibers from a liquid), wherein the polymeric coating includes a plurality of fibers arranged at or near the base forming textures (e.g., a network of textures, a network of voids) between the fibers. A technique called electro-writing is the same concept, except the filament is directed to a target surface in a controlled manner using standard xy-printing technology. In particular, in keeping with the desired Wenzel-Cassie structures, a directed electrospinning methodology can lay down individual fibers of different material precisely on top of fibers previously laid down. In at least one embodiment, the stent surface comprises alternating fibers of hydrophilic and hydrophobic materials in alternating arrangement, in stacked fashion, describing a polygonal grid, for example a rectangular grid. Such directed electrospun grid surfaces may provide a dual functionality of localizing the stent and promoting a healthy tissue in growth, wherein fibrosis is down regulated and functional muscle tissue and neovascularization is up-regulated. It is recognized that the density of such stacked polygonal mesh can promote a variety of macroscopic tissues, including, repair morphologies, kinetic morphologies (e.g., layers of muscle tissue), and pressure walling morphologies. The latter may be understood to be of particular importance in the repair of lumens that conduct fluids.

In one or more embodiments, the use of salt leaching and/or electrospinning may be used to provide a polymeric coating having one or more textures that form a network of textures (e.g., a plurality of textures in fluid communication along the base). In some embodiments, cell ingrowth may be enhanced when the polymeric coating includes a network of textures that penetrate the base, or where the base has openings into which tissue is promoted to grow. In one or more embodiments, any of a wide variety of therapeutic agents (e.g., growth factors) including, but not limited to fibronectin, and those described elsewhere herein may be included on, within, and/or in combination with a network of textures to promote tissue ingrowth when the micropatterned polymeric coating contacts tissue.

In at least one embodiment, the coating can be molded as a substantially tubular structure with a lumen defined by the base of the coating. A temporary adhesive layer or alternatively a soft hydrogel layer can be applied to either the stent or to at least a portion of the outer surface of the base of the coating. In at least one embodiment, the adhesive layer may substantially cover the entire outer surface of the base of the coating. The stent can be inserted into the lumen of the coating as a separate element. In at least one embodiment, heat and/or pressure may be applied to ensure proper adhesion of the coating to the stent via an adhesive layer. The adhesive layer may include silicone coatings, other suitable adhesives, or priming solutions that enable the coating to adhere to the metal or polymeric stent (or stent coating thereon).

In one or more embodiments, rather than being molded as a tubular structure, the coating can be molded as a strip attached to the outer surface of the stent. For example, the strip may be disposed in a helical fashion about the stent, or disposed on circumferential rings, or in counter-rotating helical configuration. In some embodiments, the strip can be applied as perimeter strips attached circumferentially about at least a portion of the circumferential perimeter of the stent. In some embodiments, the strip can be a longitudinal strip attached to the stent in a longitudinal direction. In some embodiments, the stent can be helically wrapped about the stent. In some embodiments the coating may be applied as a single strip or as multiple strips. Where the coating is applied as multiple strips, directly adjacent strips may abut one another or may be spaced apart from one another.

In at least one embodiment, the strips may be partial tubular structures that extend along the length of the stent but only cover a portion of the circumference of the stent. In some embodiments, a portion of the stent may be exposed. An adhesive layer can be applied to either the stent or to at least a portion of the base of the coating. In at least one embodiment, heat and/or pressure may be applied to ensure proper adhesion of the coating to the stent via the adhesive layer. In at least one embodiment, discrete micropatterns of micropillars can be formed on and/or attached directly to either the stent or the polymeric coating.

In one or more embodiments, the polymeric coating can be formed by dip-coating the stent in the coating material without needing an additional adhesive layer to connect the coating to the stent. For example, the stent can be inserted into a mold, which includes a cavity and a tubular member. The cavity may be defined by an inner wall of mold, which is an inverse of the desired micropattern. The stent may rest on the tubular member such that the inner surface of the stent may be disposed about the tubular member. The mold with the stent can be dipped into the coating material so that the coating material fills the mold and attaches to the stent. In some embodiments, temperature changes and/or pressure changes may be applied to the mold to cure the coating material. Once the coating material cures to form the polymeric coating, the endoprosthesis can be removed from the mold. Alternatively, the polymeric coating can be injection molded onto the stent using a similar mold. The coating material may be injected into the mold rather than the mold being dipped into the coating material.

In one embodiment, the stent may comprise an SMP. Examples of SMP's include, but are not limited to, polynorbornene and copolymers of polynorbornene, blends of polybornene with KRATON® (thermoplastic elastomer) and polyethylene, styrenic block copolymer elastomers (e.g., styrene-butadiene), polymethylmethacrylate (PMMA), polyethylene, polyurethane, polyisoprene, polycaprolactone and copolymers of polycaprolactone, polylactic acid (PLA) and copolymers of poly lactic acid, polyglycolic acid (PGA) and copolymers of polyglycolic acid, copolymers of PLA and PGA, polyenes, nylons, polycyclooctene (PCO), polyvinyl acetate (PVAc), polyvinylidene fluoride (PVDF), blends of polyvinyl acetate/polyvinylidene fluoride (PVAc/PVDF), blends of polymethylmethacrylate/polyvinyl acetate/polyvinylidene fluoride (PVAc/PVDF/PMMA) and polyvinylchloride (PVC) and blends and/or combinations thereof.

In another alternate method of manufacture, the device may be formed by molding the exterior surface modification onto a separate layer of material, such as for example a non-textile material. As used herein, the term “non-textile” and its variants may refer to a material formed by casting, molding, spinning, or extruding techniques to the exclusion of typical textile forming techniques, such as braiding, weaving, knitting and the like. Non-limiting examples of useful polymeric materials for the non-textile polymeric graft portions include, but are not limited to, polyesters, polypropylenes, polyethylenes, polyurethanes, poly naphthalene, polytetrafluoroethylene, expanded polytetrafluoroethylene, silicone, and combinations and copolymers thereof.

In some embodiments, a device as disclosed herein may be treated with a therapeutic agent or agents. “Therapeutic agents”, “pharmaceuticals,” “pharmaceutically active agents”, “drugs” and other related terms may be used interchangeably herein and include genetic therapeutic agents, non-genetic therapeutic agents, and cells. Therapeutic agents may be used singly or in combination. A wide variety of therapeutic agents can be employed in conjunction with the present invention including those used for the treatment of a wide variety of diseases and conditions (i.e., the prevention of a disease or condition, the reduction or elimination of symptoms associated with a disease or condition, or the substantial or complete elimination of a disease or condition).

Non-limiting examples of useful therapeutic agents include, but are not limited to, adrenergic agents, adrenocortical steroids, adrenocortical suppressants, alcohol deterrents, aldosterone antagonists, amino acids and proteins, ammonia detoxicants, anabolic agents, analeptic agents, analgesic agents, androgenic agents, anesthetic agents, anorectic compounds, anorexic agents, antagonists, anterior pituitary activators and suppressants, anthelmintic agents, anti-adrenergic agents, anti-allergic agents, antiamoebic agents, anti-androgen agents, anti-anemic agents, anti-anginal agents, anti-anxiety agents, anti-arthritic agents, anti-asthmatic agents, anti-atherosclerotic agents, antibacterial agents, anticholinergic agents, anticholinergic agents, anticholinergic agents, anticoagulants, anticoccidial agents, anticonvulsants, antidepressants, antidiabetic agents, antidiuretics, antidotes, antidyskinetics agents, antiemetic agents, antiepileptic agents, anti-estrogen agents, antifibrinolytic agents, antifungal agents, antiglaucoma agents, antihemophilic agents, antihemophilic Factor, antihemorrhagic agents, antihistaminic agents, antihyperlipidemic agents, antihyperlipidemic agents, antihypertensives, antihypertensives, anti-infective agents, anti-inflammatory agents, non-keratinizing agents, antimicrobial agents, antimigraine agents, antimitotic agents, antimycotic agents, antineoplastic agents, anticancer supplementary potentiating agents, antineutropenic agents, antiobsessional agents, antiparasitic agents, antiparkinsonian drugs, antipneumocystic agents, antiproliferative agents, anti-prostatic hypertrophy drugs, antiprotozoal agents, antipruritics, antipsoriatic agents, antipsychotics, antirheumatic agents, antischistosomal agents, antiseborrheic agents, antispasmodic agents, antithrombotic agents, antitussive agents, anti-ulcerative agents, anti-urolithic agents, antiviral agents, benign prostatic hyperplasia therapy agents, blood glucose regulators, bone resorption inhibitors, bronchodilators, carbonic anhydrase inhibitors, cardiac depressants, radioprotectants, cardiotonic agents, cardiovascular agents, choleretic agents, cholinergic agents, cholinergic agonists, cholinesterase deactivators, coccidiostat agents, cognition adjuvants and cognition enhancers, depressants, diagnostic aids, diuretics, dopaminergic agents, ectoparasiticides, emetic agents, enzyme inhibitors, estrogens, fibrinolytic agents, free oxygen radical scavengers, gastrointestinal motility agents, glucocorticoids, gonad-stimulating principles, hemostatic agents, histamine H2 receptor antagonists, hormones, hypocholesterolemic agents, hypoglycemic agents, hypolipidemic agents, hypotensive agents, HMG CoA reductase inhibitors, immunizing agents, immunomodulators, immunoregulators, immunostimulants, immunosuppressants, impotence therapy adjuncts, keratolytic agents, LHRH agonists, luteolysis agents, mucolytics, mucosal protective agents, mydriatic agents, nasal decongestants, neuroleptic agents, neuromuscular blocking agents, neuroprotective agents, NMDA antagonists, non-hormonal sterol derivatives, oxytocic agents, plasminogen activators, platelet activating factor antagonists, platelet aggregation inhibitors, post-stroke and post-head trauma treatments, progestins, prostaglandins, prostate growth inhibitors, prothyrotropin agents, psychotropic agents, radioactive agents, repartitioning agents, scabicides, sclerosing agents, sedatives, sedative-hypnotic agents, selective adenosine A1 antagonists, adenosine A2 receptor antagonists, serotonin antagonists, serotonin inhibitors, serotonin receptor antagonists, steroids, stimulants, thyroid hormones, thyroid inhibitors, thyromimetic agents, tranquilizers, unstable angina agents, uricosuric agents, vasoconstrictors, vasodilators, vulnerary agents, wound healing agents, xanthine oxidase inhibitors, and the like, and combinations thereof.

Useful non-genetic therapeutic agents for use in connection with the present invention include, but are not limited to, (a) anti-thrombotic agents such as heparin, heparin derivatives, urokinase, clopidogrel, and dextro phenylalanine proline arginine chloromethylketone; (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anaesthetic agents such as lidocaine, bupivacaine and ropivacaine; (e) anticoagulants such as D-Phe-Pro-Arg chloromethyl ketone, an RGD peptide-containing compound, heparin, hirudin, antithrombin compounds, platelet receptor antagonists, antithrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin; (t) smooth muscle relaxants such as alpha receptor antagonists (e.g., doxazosin, tamsulosin, terazosin, prazosin and alfuzosin), calcium channel blockers (e.g., verapamil, diltiazem, nifedipine, nicardipine, nimodipine and bepridil), beta receptor agonists (e.g., dobutamine and salmeterol), beta receptor antagonists (e.g., atenolol, metoprolol and butoxamine), angiotensin-II receptor antagonists (e.g., losartan, valsartan, irbesartan, candesartan, eprosartan and telmisartan), and antispasmodic/anticholinergic drugs (e.g., oxybutynin chloride, flavoxate, tolterodine, hyoscyamine sulfate, dicyclomine); (u) bARKct inhibitors; (v) phospholamban inhibitors; (w) Serca 2 gene/protein; (x) immune response modifiers including aminoquinolines, for instance, imidazoquinolines such as resiquimod and imiquimod; (y) human apolioproteins (e.g., AI, AII, AIII, AIV, AV, etc.); (z) selective estrogen receptor modulators (SERMs) such as raloxifene, lasofoxifene, arzoxifene, miproxifene, ospemifene, PKS 3741, MF 101 and SR 16234; (aa) PPAR agonists, including PPAR-alpha, gamma and delta agonists, such as rosiglitazone, pioglitazone, neto glitazone, fenofibrate, bexarotene, metaglidasen, troglitazone and tesaglitazar; (bb) prostaglandin E agonists, including PGE2 agonists, such as alprostadil or ONO 8815Ly; (cc) thrombin receptor activating peptide (TRAP); (dd) vaso peptidase inhibitors including benazepril, fosinopril, lisinopril, quinapril, ramipril, imidapril, delapril, moexipril and spirapril; (ee) thymosin beta 4; (ff) phospholipids including phosphorylcholine, phosphatidylinositol and phosphatidylcholine; and (gg) VLA-4 antagonists and VCAM-1 antagonists. The non-genetic therapeutic agents may be used individually or in combination, including in combination with any of the agents described herein.

Further examples of non-genetic therapeutic agents, not necessarily exclusive of those listed above, include taxanes such as paclitaxel (including particulate forms thereof, for instance, protein-bound paclitaxel particles such as albumin-bound paclitaxel nanoparticles, e.g., ABRAXANE), sirolimus, everolimus, tacrolimus, zotarolimus, Epo D, dexamethasone, estradiol, halofuginone, cilostazol, geldanamycin, alagebrium chloride (ALT-711), ABT-578 (Abbott Laboratories), trapidil, liprostin, Actinomycin D, Resten-NG, Ap-17, abciximab, clopidogrel, Ridogrel, beta-blockers, bARKct inhibitors, phospholamban inhibitors, Serca 2 gene/protein, imiquimod, human apolipoprotein, growth factors (e.g., VEGF-2), as well derivatives of the foregoing, among others.

Biodegradable polymers that can be used to form a device as disclosed herein, to form at least a part of a device as disclosed herein, or can be coated onto a device or part of a device as disclosed herein, may include a wide variety of materials. Examples of such materials, include but are not limited to, polyesters, polylactides, polycarbonates, polyanhydrides, poly(amino acids), polyimines, polyphosphazenes and various naturally occurring biomolecular polymers, as well as co-polymers and derivatives thereof. Certain hydrogels, which are cross-linked polymers, can also be made to be biodegradable. These include, but are not necessarily limited to, polyesters, pluronans, poly(amino acids), copoly(ether-esters), polyalkylenes oxalates, polyamides, poly(imino carbonate), polyorthoesters, polyoxaesters, polyamidoesters, polyoxaesters containing amido groups, polyanhydrides, polyphosphazenes, poly-alpha-hydroxy acids, trimethylene carbonate, poly-beta-hydroxy acids, polyorganophosphazines, polyanhydrides, polyesteramides, polyethylene oxide, polyester-ethers, polyphosphoester, polyphosphoester urethane, cyanoacrylates, poly(trimethylene carbonate), poly(imino carbonate), polyalkylene oxalates, polyvinylpyrrolidone, polyvinyl alcohol, poly-N-(2-hydroxypropyl)-methacrylamide, polyglycols, aliphatic polyesters, poly(orthoesters), poly(ester-amides), polyanhydrides, modified polysaccharides and modified proteins. Some specific examples of bioabsorbable materials include poly(epsilon-caprolactone), poly(dimethyl glycolic acid), poly(hydroxybutyrate), poly(p-dioxanone), polydioxanone, PEO/PLA, poly(lactide-co-glycolide), poly(hydroxybutyrate-co valerate), poly(glycolic acid-eo-trimethylene carbonate), poly(epsilon-caprolactone-co-p-dioxanone), poly-L′Glutamic acid or poly-L-Lysine, polylactic acid, polylactide, polyglycolic acid, polyglycolide, poly(D,L-lactic acid), L-polylactic acid, poly(glycolic acid), polyhydroxyvalerate, cellulose, chitin, dextran, fibrin, casein, fibrinogen, starch, collagen, hyaluronic acid, hydroxyethyl starch, and gelatin.

The above disclosure is intended to be illustrative and not exhaustive. This description will suggest many variations and alternatives to one of ordinary skills in this art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the present disclosure such that the present disclosure should be recognized as also specifically directed to other embodiments having any other possible combination of the features of the dependent claims.

Thus, although there have been described particular embodiments of the present invention of a new and useful ANTI-FOULING STENT it is not intended that such references be construed as limitations upon the scope of this invention except as set forth in the following claims.

What is claimed is:

1. An endoprosthesis for placement in a body lumen comprising:
a tubular member having an outer surface and an inner surface, the outer surface comprising a first hierarchical microstructure pattern that is arranged to generate a Wenzel-Cassie state, the inner surface being superhydrophobic or oleophobic and comprising a second microstructure pattern that is different than the first hierarchical microstructure pattern.

2. The endoprosthesis of claim 1, wherein the tubular member further comprises a polymer wall such that the outer surface and inner surface are positioned on opposing sides of the polymer wall, and wherein at least one through-hole fluidly connects the outer surface and the inner surface.

3. The endoprosthesis of claim 1, wherein the first hierarchical microstructure pattern comprises a plurality of first microfeatures having a base and a top portion wherein each of the first microfeatures of the plurality of first microfeatures include the base having a recurved pillar.

4. The endoprosthesis of claim 3, wherein the first hierarchical microstructure pattern comprises a plurality of second microfeatures disposed about the top portion of the plurality of first microfeatures.

5. The endoprosthesis of claim 4, wherein the plurality of first microfeatures may have a pitch between adjacent microfeatures from 25-100 microns and a height from 10-100 microns.

6. The endoprosthesis of claim 1, wherein the second hierarchical microstructure pattern comprises a plurality of microridges wherein each microridge of the plurality of microridges has a pitch between adjacent microridges from 100 nm to 10 microns.

7. The endoprosthesis of claim 6, wherein each microridge of the plurality of microridges comprise a length that is greater than a width.

8. The endoprosthesis of claim 7, wherein the plurality of microridges comprises a subset of microridges wherein a height of each adjacent microridge increases progressively.

9. The endoprosthesis of claim 8, wherein the plurality of microridges comprises a second subset of microridges wherein a height of each adjacent microridge decreases progressively.

10. The endoprosthesis of claim 9, wherein the first subset of microridges is adjacent the second subset of microridges.

11. The endoprosthesis of claim 7 wherein the length of each microridge of the plurality of microridges is arranged coaxially with a central axis of the tubular member.

12. The endoprosthesis of claim 7 wherein the length of each microridge of the plurality of microridges is arranged circumferentially along the inner surface of the tubular member.

13. The endoprosthesis of claim 1, wherein the first hierarchical microstructure covers a portion of the outer surface.

14. The endoprosthesis of claim 1, wherein the outer surface comprises a third microstructure pattern different from the first microstructure pattern, configured to transport fluid from a first location to a second location.

15. The endoprosthesis of claim 14, wherein the third microstructure pattern comprises a plurality of microfeatures having a pitch between adjacent microfeatures from 5-50 microns, a width from 1-20 microns, and a height from 5-50 microns.

16. The endoprosthesis of claim 1, wherein the tubular member comprises a first end and a second end, the first end and second end being flared in relation to a central portion of the tubular member.

17. An endoprosthesis for placement in the gastrointestinal tract comprising:
a tubular member having an outer surface and an inner surface, the outer surface comprising a first hierarchical microstructure pattern that is multi-fractal, the outer surface additionally comprising a plurality of pores, and the inner surface being superhydrophobic or oleophobic and comprising a second microstructure pattern that is different than the first hierarchical microstructure pattern.
